(19) United States
(12) Patent Application Publication
(10) Pub. No.: US 2023/0009299 A1
(43) Pub. Date: Jan. 12, 2023

(54) Tiffanye's Natural Hair Growth Oil

(71) Applicant: Tiffanye Michelle Hopkins, Clarksville, TN (US)

(72) Inventor: Tiffanye Michelle Hopkins, Clarksville, TN (US)

(21) Appl. No.: 17/745,836

(22) Filed: May 16, 2022

Related U.S. Application Data
(63) Continuation of application No. 63/218,910, filed on Jul. 7, 2021.

(51) Int. Cl. A61K 8/92; A61Q 7/00

(52) U.S. Cl. CPC A61K 8/922; A61Q 7/00

(57) ABSTRACT

Shake Well. Apply a few drops once a week or as needed on damp or dry hair, massage well, and style as usual.

Precaution: if this product causes any irritation, please stop using it. If the product becomes clumpy or appears solid just emerged in hot water before use.

### HAIR INGREDIENTS

Black Castrol Oil
Sweet Almont oil
Peppermint oil
Grape seed oil
Neem oil
Avocado Oil
Vitamin E Oil

Alternate oil if needed to substitute:
Jojoba oil
Tea tree oil
Eucalyptus oil
Coconut oil

Combine 1 oz of grapeseed oil, 1 oz of almond oil, 1 oz of avocado oil, and 1 oz of black castor oil; then stir in 2 oz of neem oil, ½ dropper of vitamin E oil, and 3 drops of peppermint oil. Mix well using a whisk. This makes one 1 oz bottle.

Combine 2 oz of grapeseed oil and avocado oil, 2½ oz of almond oil and black castor oil, 1½ oz of neem oil, 3 (½) droppers of vitamin E oil, and 10 drops of peppermint oil. Mix well with a whisk. This makes eight or ten 1 oz bottles.

The alternative oils are used if someone has an allergy to any of the oils above. This is used only if I need to substitute an oil.

1½ oz of coconut oil

3 drops of Eucalyptus oil

3 drops of tea tree oil

1. I Tiffanye Hopkins claim as the sole ownership and inventor of Tiffanye's Natural Hair Growth Oil.
